(19) United States
(12) Patent Application Publication
(10) Pub. No.: US 2010/0206316 A1
(43) Pub. Date: Aug. 19, 2010

(54) METHOD FOR DETERMINING CHROMOSOMAL DEFECTS IN AN IVF EMBRYO

(76) Inventors: Richard T. Scott, JR., Far Hills, NJ (US); Nathan R. Treff, Chester, NJ (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 12/690,665

(22) Filed: Jan. 20, 2010

Related U.S. Application Data
(60) Provisional application No. 61/205,522, filed on Jan. 21, 2009.

(51) Int. Cl. A61B 17/00; C12Q 1/68; C40B 40/06; C40B 50/18

(52) U.S. Cl. 128/898; 435/6; 506/16; 506/32

(57) ABSTRACT

The present invention is directed to methods for determining the presence or absence of a genetic defect in an IVF embryo prior to transfer comprising identifying a set of informative SNPs in the genotype of the embryo's parents; assaying the genotype of two or more informative SNPs from the set of informative SNPs on one or more chromosomes collected from a cell of the embryo; determining the presence or absence of a genetic defect in the embryo based on the genotype of the two or more informative SNPs on one or more chromosomes of the embryo; and selecting a candidate IVF embryo determined to be without genetic defect for transfer.

### CROSS-REFERENCE TO RELATED APPLICATIONS

This application claims the benefit of the filing date of U.S. Provisional Patent Application No. 61/205,522 filed Jan. 21, 2009, the disclosure of which is hereby incorporated herein by reference.

The instant application contains a Sequence Listing which has been submitted via EFS-Web and is hereby incorporated by reference in its entirety. Said ASCII copy, created on Jan. 13, 2010, is named RMA30002.txt, and is 3,042 bytes in size.

### BACKGROUND OF THE INVENTION

For several decades many couples have been treated for infertility using the technique of in vitro fertilization (IVF). This procedure involves the in vitro incubation of sperm and an egg in culture media in which fertilization takes place. The fertilized egg is then cultured in special media for several days before the embryo is transferred into the female patient.

Typically, embryos are cultured for 3 days prior to transfer. It is also clinically possible to culture IVF embryos for several more days during which time the embryo develops into a blastocyst. Delaying embryo transfer until day 5 is thought to result in a greater chance of implantation, thus clinicians need not transfer as many embryos as might be typically transferred on day 3, thus reducing the possibility of a high risk multiple pregnancy. In some cases, embryos may be transferred on day 6, as some blastocysts may develop more slowly than others, but are still reproductively competent.

Preimplantation genetic diagnosis (PGD) may be used to screen IVF embryos for genetic defects, or otherwise grade the embryo's viability, prior to embryo transfer. Of the possible genetic defects, aneuploidy is the most prevalent genetic abnormality in human embryos derived through in vitro fertilization. By identifying embryos with chromosomal abnormalities such as aneuploidy, PGD can be used to avoid transferring unhealthy embryos which may fail to implant or which may eventually end in a miscarried pregnancy. Using PGD to determine the presence of chromosomal abnormalities in an IVF embryo prior to transfer can also ease the minds of individuals with a family history of genetic disease and who fear passing on a genetic abnormality to their child.

PGD involves the analysis of nucleic acid derived from cells removed from an IVF embryo during the preimplantation stage of development. While biopsy of first polar bodies prior to fertilization or second polar bodies after fertilization on day 1 is possible, typically, PGD is performed using nucleic acid isolated from a single cell from a day 3 embryo. One or more healthy embryos identified by genetic analysis can then be transferred. If the embryo is to be transferred before day 5 (or day 6, in some cases), the embryos need not be frozen.

US 2008/0243398 and related application, 2007/0184467 (Rabinowitz et al.) describe a mathematical protocol for cleansing noisy genetic data and determining chromosome copy number. The techniques disclosed involve assay of the genotype of one or more fertilized embryos as well as of the parents or other related individuals. Through sophisticated mathematical filtering, the genomes are compared in order to reconstruct the incomplete genetic data obtained from the embryo with the data obtained from the parents or related individuals to permit analysis of chromosome copy number in the embryo or to make phenotypic predictions. However, this technique involves whole genome analysis of the embryo, parents and/or other related individuals, the creation of data which may contain significant amplification errors, as well as the mathematical manipulation of a considerable volume of data. (See also, Johnson, D. S. et al., Fertility and Sterility, Vol. 90, Suppl 1, September 2008, pp. S309-S310; Rabinowitz, M. et al., Fertility and Sterility, Vol. 90, Suppl 1, September 2008, p. S23; and Johnson, D. M. et al., Fertility and Sterility, Vol. 89, Issue 4, p. S5).

U.S. Pat. No. 7,442,506 and U.S. Pat. No. 7,332,277 disclose methods for screening a fetus at multiple loci of interest associated with a trait or disease state to detect genetic disorders in a fetus.

### BRIEF SUMMARY OF THE INVENTION

In one aspect, the present invention is directed to a method for determining the presence or absence of a genetic defect in an IVF embryo prior to transfer comprising identifying a set of informative SNPs from the genotype of the embryo's parents; assaying the genotype of two or more informative SNPs from the set of informative SNPs on one or more chromosomes collected from a cell of the embryo; determining the presence or absence of a genetic defect in the embryo based on the genotype of the two or more informative SNPs on one or more chromosomes of the embryo; and selecting a candidate IVF embryo determined to be without genetic defect for transfer.

In various embodiments, the genetic defect is a mutation in the DNA of the embryo, chromosomal abnormality and/or aneuploidy, for example, a genetic defect as provided in detail in Table 1 of the disclosure, and/or a form of aneuploidy selected from the group consisting of nullisomy, monosomy, disomy, trisomy, and tetrasomy.

In an additional embodiment, the embryo is genotyped comprising using a subset (e.g., less than all) of the set of informative SNPs identified in the genotype of the embryo's parents.

In various embodiments, the step of identifying a set of informative SNPs comprises determining one or more homozygous opposite SNPs on one or more chromosomes of the embryo's parents and/or determining one or more heterozygous SNPs on one or more chromosomes of the embryo's parents.

In another embodiment, the step of determining the presence or absence of a genetic defect in the embryo comprises genotyping two or more informative SNPs on a plurality of chromosomes of the embryo and/or genotyping two or more informative SNPs on all of the chromosomes of the embryo.

In various additional embodiments, the IVF embryo to be assayed according to the methods of the present invention is a human embryo, and may be a day 3, day 4, day 5 or day 6 embryo.

As contemplated herein, in a further embodiment, the informative SNPs to be genotyped according to the methods of the present invention are located on chromosomes selected from the group consisting of chromosomes 1, 2, 3, 4, 5, 6, 7, 8, 9, 10, 11, 12, 13, 14, 15, 16, 17, 18, 19, 20, 21, 22, X, and Y. In a particular embodiment, the chromosomes are chromosomes 13, 18, and 21.

In an additional embodiment, the methods of the present invention further comprise transferring the selected candidate IVF embryo on the same day as the steps of assaying, determining and selecting. In particular embodiments, the assaying, determining, selecting and transferring of the IVF embryo are accomplished within about 24 hours, within about 12 hours or less, or within about 8 hours or less.

In another embodiment, the methods of the invention further comprise assaying more than one cell from the IVF embryo. In a particular embodiment, the IVF embryo is a blastocyst. In a further embodiment, the cells are biopsied from trophoectoderm. In yet an additional embodiment, the assaying, determining, selecting and transferring of the blastocyst are accomplished within about 24 hours, within about 12 hours or less, or within about 8 hours or less.

In yet additional embodiments, three or less IVF embryos are transferred, two or less IVF embryos are transferred, or one IVF embryo is transferred.

In yet another embodiment, the informative SNPs are identified using, among other things, one or more database(s) of genetic information. In various further embodiments, the database(s) contain(s) information regarding the genetic diversity characteristic of the human genome, or information regarding the genetic diversity characteristic of a particular racial, ethnic, religious or geographic group. In an additional embodiment, the database contains information regarding diallelic SNPs with a relatively high minor allele frequency. In a particular embodiment, the high minor allele frequency is from about 40% to less than about 50%.

In further embodiments, the methods of the invention further comprise selecting. about 1000 or less, about 500 or less, about 400 or less, about 250 or less, about 100 or less, or about 50 or less homozygous opposite SNPs from the set of informative SNPs to generate a subset of informative SNPs for genotyping the one or more chromosomes of the embryo.

In additional embodiments, the subset of informative SNPs comprises about 100 or less, about 50 or less, about 40 or less, or about 20 or less SNPs per chromosome.

In yet more embodiments, the subset of informative SNPs may comprise at least three, at least five, or at least ten informative SNPs.

In another aspect, the invention relates to a method for transferring an IVF embryo comprising identifying informative SNPs by genotyping SNPs on a plurality of chromosomes of the parents to identify homozygous opposite SNPs (i.e., a set of informative SNPs); assaying the genotype of one or more of the identified informative SNPs on a plurality of chromosomes collected from a cell of the embryo; determining the presence or absence of a genetic defect in the embryo based on the genotype of the informative SNPs in the embryo; and transferring the embryo if determined to be without genetic defect within about 154 hours of fertilization, or between about 48 and about 144 hours of fertilization.

In various embodiments, the assaying, determining and transferring steps are accomplished within a period of about 48, about 24, about 16, about 12 or about 8 hours.

In yet another embodiment, only a subset of informative SNPs identified by genotyping the parents is genotyped in the embryo. In an additional embodiment, the same number of informative SNPs genotyped in the parents is genotyped in the embryo.

In another aspect, the invention relates to a method for preimplantation genetic diagnosis and fresh transfer of a day 3, day 4, day 5 or day 6 IVF embryo comprising identifying informative SNPs by genotyping SNPs on a plurality of chromosomes of the parents to identify homozygous opposite SNPs; assaying the genotype of one or more of the informative SNPs in the embryo on a plurality of chromosomes of the IVF embryo; determining the presence or absence of a genetic defect in the embryo based on the genotype of the informative SNPs in the embryo; and transferring the embryo if determined to be without genetic defect within about 24 hours of the assaying step.

In a further aspect the invention is directed to arrays of potentially informative SNPs or informative SNPs comprising a plurality of nucleic acid probes comprising nucleic acid for one or more SNPs from one or more human chromosomes wherein the SNPs are homozygous opposites of high minor allele frequency. In a particular embodiment, the probes are immobilized on a solid support. In an additional embodiment, the nucleic acid in the array comprises at least two SNPs from one or more of human chromosomes 1-22, X and Y.

In another aspect, the invention relates to methods for making an array for preimplantation genetic diagnosis of an IVF embryo comprising identifying informative SNPs for preimplantation genetic diagnosis, selecting an informative SNP for at least one chromosome, and affixing nucleic acid probes for the informative SNP on a solid support.

In another aspect, the invention is directed to kits comprising an array of nucleic acid probes immobilized on a solid support, the array comprising nucleic acid probes for one or more SNPs from one or more human chromosomes wherein the SNPs are informative SNPs for determining the presence or absence of a genetic defect in an IVF embryo prior to transfer according to the methods of the present invention.

### BRIEF DESCRIPTION OF THE DRAWINGS

FIG. 1 is a graph illustrating the ability to discriminate monosomy or trisomy from disomy for chromosome 21 using SNP genotyping and allele copy number analysis for an informative SNP (where expected genotype is heterozygous) in single cells with known copy number of chromosome 21.

FIG. 2 is a plot of signal intensities for FGFR3 allele A and G in a child with dwarfism, parents, and an unrelated control using five cells.

### DETAILED DESCRIPTION

While the specification concludes with the claims particularly pointing and distinctly claiming the invention, it is believed that the present invention will be better understood from the following description.

All percentages and ratios used herein are by weight of the total composition and all measurements made are at 25° C. and normal pressure unless otherwise designated. All temperatures are in Degrees Celsius unless specified otherwise. The present invention can comprise (open ended) or consist essentially of the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise.

All ranges recited herein include the endpoints, including those that recite a range “between” two values. Terms such as “about,” “generally,” “substantially,” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value. Unless otherwise indicated, as used herein, “a” and “an” include the plural, such that, e.g., “a cell” can mean more than one cell.

By applying quantitative statistical analysis and Mendelian inheritance rules, various combinations of parental SNP genotypes may be informative for diagnosing chromosomal abnormalities in an IVF embryo. Particularly, analysis of diallelic single nucleotide polymorphisms (SNPs) can be used to evaluate the ploidy status of the chromosome in which the SNP is found and/or to determine the presence of other genetic abnormalities in the IVF embryo.

SNPs are single base pair positions in DNA at which different alleles, or alternative nucleotides, exist in the genome between members of a species or chromosome pair in an individual. An individual may be homozygous or heterozygous for an allele at each SNP position. For example, with regard to organisms that have two copies of each chromosome, if both chromosomes have the same allele, the condition is referred to as homozygous. If the alleles at the two chromosomes are different, the condition is referred to as heterozygous.

A SNP may arise due to a substitution of one nucleotide for another at the polymorphic site. They may be bi-, tri-, or tetra- allelic, although tri- and tetra-allelic polymorphisms are extremely rare, almost to the point of non-existence (Brookes, Gene 234 (1999) 177-186). For this reason, SNPs are often referred to as “bi-allelic markers” or “di-allelic markers”. Of the two alleles, typically one is observed in a population at a lower frequency than the other and this is referred to as the minor allele frequency (less than 50%).

As used herein, the term “informative SNP” (also referred to herein as an “allele of interest”) refers to SNPs which may be genotyped in the embryo to reveal information regarding the genetic condition of the embryo. For example, in a particular embodiment of the present invention, “potentially informative SNPs”, are diallelic SNPs that are characterized in a SNP database as having a high minor allele frequency. As used herein, “high minor allele frequency” is a frequency less than 50%, typically a frequency greater than 40% but less than 50%. By selecting such SNPs for assay, there is a high likelihood that the parents of an IVF embryo will in fact be homozygous opposite at that allele, and the resulting embryo, if normal, will be heterozygous for this SNP. If after assay, the parents are, in fact, homozygous opposites at that allele, then that SNP is an “informative SNP”.

Proper embryonic allele ratios (1:1) may be predicted by evaluating parental genotypes and utilizing statistical analysis and Mendelian inheritance rules. Thus, a SNP in which each parent is homozygous for the opposite allele is informative for both monosomy and trisomy in an IVF embryo. For example, if the mother is homozygous AA and the father is homozygous TT (each parent has two alleles for each SNP, one each on each chromosome), than all embryos should inherit an AT genotype (one allele from each parent) if that embryo is euploid for the chromosome in which that SNP is located. The allele ratio for a normal embryo for a SNP where the parents are homozygous for the opposite allele will be 1:1. That is, one copy of A from the mother and 1 copy of T from the father. However, if the embryo is monosomy for that chromosome then the genotype will be A_{—} or T_{—} (hemizygous), and the allele ratio will be either 0:1 or 1:0. In contrast, if the embryo is trisomy, then the allele ratio will be 2:1 or 1:2 (AAT or TTA).

In another embodiment it is also contemplated herein that informative SNPs for preimplantation genetic diagnosis according to the methods of the present invention include SNPs for which both parents are heterozygous. For example, if the mother and father are both AT for a particular SNP, a euploid embryo will have an allele ratio of either AT (1:1), AA (1:1), or TT (1:1). In this case, an IVF embryo with trisomy will either be AAT (2:1) or ATT (1:2). It is also possible in some embodiments that the set of informative SNPs for which both parents are homozygous be supplemented with other SNPs for which both parents are heterozygous. For example, if parental genotyping reveals a statistically inadequate number of homozygous opposite SNPs on a chromosome, SNPs on the chromosome for which the parents are heterozygous may be included in genotyping an embryo. This would be particularly useful for assessing an embryo for trisomy. Optionally, even if a statistically acceptable number of homozygous opposite SNPs are identified per chromosome, genotyping embryos at SNPs for which both parents are heterozygous may be performed to produce a more robust data set. In this case, all of the SNPs which are tested from the parents and which are either homozygous opposites or heterozygous form the set of informative SNPs. Those SNPs selected from that set for use in genotyping the embryo, which may be all of the informative SNPs, but is often fewer, is called a subset of informative SNPs. As contemplated herein, by evaluating parental genomic DNA, one may establish characteristic profiles of nullisomy, monosomy, disomy, trisomy, and tetrasomy. With this data, the ploidy status of embryonic chromosomes may be evaluated. Indeed, by measuring the allele ratio for multiple informative SNPs across all 24 chromosomes, one can establish a full karyotype (number of chromosomes) for any embryo.

In addition to screening for aneuploidy in an IVF embryo, the methods of the present invention may be used in PGD to identify IVF embryos containing genetic predisposition to diseases related to single point mutations. These diseases may be detected by screening for the presence or absence of mutant alleles in parental and/or embryonic nucleic acid. For example, in situations where one or both parents are carriers, or heterozygous, for a disease-related mutant allele, nucleic acid from embryos may be screened to determine whether the embryo has inherited the mutation. In this case, an informative SNP is a SNP that is associated with a particular genetic defect.

Potentially informative SNPs for use in the methods of the present invention may be identified by reviewing publicly or commercially available genetic databases to identify SNPs that occur in a population at a frequency that will produce statistically useful data for PGD. For example, to detect monosomy or trisomy, potentially informative SNPs are SNPs that occur at a frequency such that the chances are good that the parents of the IVF embryo will be homozygous for the opposite allele. In this case, the chances that a SNP will be informative can be improved by selecting SNPs that have a high minor allele frequency in the population as it increases the chance that the parents will be homozygous opposites. In another example, to detect trisomy, informative SNPs are SNPs that occur at a frequency such that the chances are good that the parents of the IVF embryo will be heterozygotes. Such frequency may be about 30% or greater, for example, about 40%.

As used herein, the term “database of genetic information” includes databases characterizing SNP data for various populations and includes, for example, the Entrez SNP database available from NCBI, as well as databases available from NCI, WICGR, HGBASE or the International HapMap Project (see, e.g., International HapMap Consortium, Nature 449, 18 October 2007, 851-862). Extensive proprietary SNP databases are also available through commercial vendors, and include, for example, Applied Biosystems' SNP database which supports their commercial TaqMan® SNP Genotyping Assays.

While any number of potentially informative SNPs and ultimately informative SNPs may be used according to the methods of the present invention, sets of a limited number of informative SNPs may be identified and further characterized into subsets of informative SNPs. As used herein, a set of potentially informative SNPs typically refers to a group of possible informative SNPs which are of interest for the purpose of performing PGD on an IVF embryo. The set may contain hundreds of thousands of SNPs, but as contemplated herein, the methods of the present invention do not require the evaluation of a volume of data such as might be generated from a whole genome analysis. Thus, a set of potentially informative SNPs may comprise less data than the entire genotype of an individual and be of a limited number of, e.g., less than about 100,000, less than about 50,000, less than about 20,000, less than about 10,000, less than about 5,000, or less than about 1,000 potentially informative SNPs. Once these potentially informative SNPs are assayed in the parents, the SNPs which are in fact homozygous opposites (and/or as appropriate, heterozygous) may be identified, and included in a set of informative SNPS.

It is further contemplated herein that PGD of an embryo according to the methods disclosed herein may be performed using a subset of informative SNPs identified in the genotype of the embryo's parents. Smaller sets of informative SNPs, or “subsets”, may be compiled from a set of informative SNPs for use according to the methods of the present invention. Subsets may be selected for use in the methods of the present invention based on various criteria, including but not limited to, known disease association, the number of SNPs necessary to produce statistically significant data, the robustness of the chemistry and other technical factors associated with assaying a particular SNP, e.g., availability of primers or other factors associated with amplifying a particular SNP, costs associated with use of a particular SNP, and performance on samples with known karyotypes. In addition, a statistically sufficient number of informative SNPs per chromosome (including number per short and long arms), and for a statistically sufficient number of chromosomes, should be selected for assay. These factors are also relevant with regard to the selection of potentially informative SNPs. Thus, out of a potential pool of homozygous opposite (and/or heterozygous) informative SNPs identified for a particular couple, only a select subset may be used to perform PGD of an IVF embryo according to the methods of the present invention.

Sets of potentially informative SNPs may be used in the construction of a universal array of relatively few (e.g., less than about 20,000, in some embodiments less than about 10,000, in some embodiments less than about 2,000, and in other embodiments less than about 1,000, e.g., about 1000 or less, about 500 or less, about 400 or less, about 250 or less, about 100 or less or about 50 or less) alleles for genotyping the parents. In some embodiments, the set of potentially informative SNPs comprises at least about 100 or less, about 50 or less, about 40 or less, or about 20 or less SNPs per chromosome. The set of potentially informative SNPs may comprise at least 3 SNPs, at least 5 SNPs or at least 10 SNPs.

By focusing on only certain alleles, a high quality set of informative SNPs may be obtained and used (in whole or in part in the form of subset(s) thereof) to analyze an embryo. Employing a set of informative SNPs that is equal to or less than the number of potentially informative SNPs used to analyze the parents in combination with high throughput analysis (e.g., real time PCR), more accurate data may be obtained and in less time than conventional analyses. In addition, by employing the methods of the present invention for PGD, one may avoid the less efficient and error prone method of whole genome embryonic analysis which necessitates supplementary analysis of parental DNA to confirm embryonic data and/or to identify additional informative loci for embryonic genotyping.

Unlike PGD techniques that require the analysis of the entire genome, the present invention permits the analysis of a select number of alleles in the parents' genotype and allows for a further reduction in number of SNPs that need be analyzed in each embryo. This permits the practical application of real time PCR to the analysis of the genotype of each of the SNPs and the robust qualitative and quantitative assessment in real time of each reaction. This approach results is the genotypic analysis of fewer, but more informative, alleles in a shorter period of time and with greater accuracy. Furthermore, the methods of the present invention permit PGD and fresh transfer of an IVF embryo while avoiding the need to employ accelerated protocols which require the sacrifice of data quality for turn-around time such as described in US 2008/0243398.

As contemplated herein, the robust methods of the present invention permit the method steps of assaying the genotype of an embryo, determining the presence or absence of genetic defect, and selecting and transferring an embryo to be performed within a period of about 24 hours or less, e.g., within about 16, about 12, or about 8 hours or less. As such, same day cell biopsy and fresh transfer of an IVF embryo is possible. According to the methods of the present invention, it is further contemplated that the method steps may be performed such than an embryo is determined to be without genetic defect and transferred within about 154 hours of fertilization, particularly between about 48 and about 144 hours of fertilization.

As described above, sets of potentially informative SNPs as well as sets and subsets of informative SNPs may be used in PGD according to the methods of the present invention. As contemplated herein, nucleic acid probes encoding such SNPs can be provided in the form of an array for analysis. Such arrays are familiar to one of skill in the art and may be in various forms, including but not limited to, a solid support such as a chip or glass slide, e.g., in the form of a microarray or other assay plate, to which the nucleic acid may be affixed according to methods familiar to one of skill in the art. Custom made assay chips with nucleic acid for alleles of interest affixed thereto may be obtained from commercial vendors, e.g., Applied Biosystems Inc. (Foster City, Calif.), Affymetrix Inc. (Santa Clara, Calif.), or Illumina Inc. (San Diego, Calif.). This chip may be used to identify informative SNPs in the parents' genotype before being used to genotype the embryo. For example, in one embodiment, the chip is designed to contain nucleic acid for SNPs known to have high minor allele frequency in the general population, with the expectation that use of the chip will likely identify a statistically useful number of homozygous opposite SNPs in the parent's genotypes. Such chip is referred to herein as a “universal array”. A “statistically useful number” means at least 2 and frequently about 3, about 5, about 10, or about 20 or more informative SNPS per chromosome, as the situation requires.

If a statistically inadequate number of homozygous opposite SNPs are initially detected in the parents, one or more additional chips containing nucleic acid for different potentially informative SNPs may be used to screen parental nucleic acid for additional informative SNPs. As contemplated herein, one may use all of the informative SNPs identified, or a subset or subsets thereof, to genotype the embryo. Subsets of informative SNPs may be selected based on the various criteria discussed above and subsets of varying size and SNP identity are contemplated.

In addition, as discussed above, depending on the nature of the genetic defect being investigated, informative SNPS which are homozygous opposites may be supplemented with alleles for which the parents are heterozygous and the embryo may be genotyped using all or a subset or subsets of these informative homozygous opposite SNPS and the heterozygous SNPs as well. These could all be located on a single chip or different chips, or the analysis could be run on individual SNPs without using a chip based assay system. Variations of this approach apparent to one of skill in the art are also contemplated herein.

While sets of potentially informative SNPs may be identified and affixed to chips to create a “universal array” for use in the methods of the present invention, the frequency of a particular SNP may vary from one group of humans to another. Thus, it is understood that, in addition to potentially informative SNPs for a “universal array”, additional sets of potentially informative SNPs may be selected by one of skill in the art which may more closely reflect the genetic diversity of the prospective parents of an IVF embryo, e.g., with regard to race, religion, ethnicity or geographic group. Sets of potentially informative SNPs for a particular group (“group array”) may thus provide a richer data set (i.e., a data set more likely to provide informative SNPs) for use with the methods of the present invention than use of a universal array.

While universal arrays and group arrays of potentially informative SNPs may be used to genotype the parents of an IVF embryo to identify sets and subsets of informative SNPs, some parental genotypes may not demonstrate sufficient allelic homozygosity when screened against a universal array or group array. This may occur when the frequency of a minor allele of a diallelic SNP is so low that very few individuals tend to be homozygous for that allele. In this case, one may create a “custom array” for genotyping an IVF embryo by directly evaluating parental genomic DNA to identify a set of informative SNPs for this couple (e.g., SNPs for which the parents are confirmed homozygous for the opposite allele). As provided above, the embryo may then be assayed using the set of informative SNPs and/or subsets thereof.

As understood herein, informative SNPs may be identified and used to create assay chips or other tools of molecular biology such that the methods of the present invention may be performed using high throughput genomic analysis. For example, by employing conventional methods in combination with the teachings provided herein, once might make such an array by first identifying informative SNPs for preimplantation genetic diagnosis, selecting informative SNPs for at least one chromosome, and affixing nucleic acid probes for the informative SNPs on a solid support or in other suitable forms familiar to one of skill in the art.

SNPs may also be evaluated in multiple, parallel, independent genomic analyses according to conventional methods, for example, when smaller numbers or small subsets, of potentially informative SNPs, or informative SNPs, are to be assayed. One of skill in the art would be able to discern the appropriate methodology to employ for genotyping a given number of SNPs.

Further contemplated herein are kits comprising arrays for use with the methods of the present invention. For example, a kit might comprise an array of nucleic acid probes immobilized on a solid support, the array comprising nucleic acid probes for one or more SNPs from one or more human chromosomes wherein the SNPs are informative SNPs for determining the presence or absence of a genetic defect in an IVF embryo prior to transfer according to the methods discuss herein. Additional components of such kits may comprise instructions as well as reagents, primers, probes or other tools of molecular biology familiar to one of skill in the art that might be of use in conducting PGD of an IVF embryo.

In order to facilitate the PGD and fresh transfer of an IVF embryo according to the methods of the present invention, and in the time frame described, it is contemplated herein that the identification of informative SNPs by genotypic analysis of persons seeking to become parents through in vitro fertilization may occur prior to the actual creation of an IVF embryo for the couple. In this way, parental data necessary to perform the methods of the present invention may be already on hand when an IVF embryo becomes available for PGD.

In addition, one of skill in the art will appreciate that the steps of the methods of the present invention may take place in different locations. For example, biological material may be obtained from the prospective parents of an IVF embryo and used to create an IVF embryo at the same clinic, or the materials may be transported according to conventional methods to a second location at which the IVF embryo may be created and maintained in vitro. Similarly, cells may be obtained from an IVF embryo and screened for PGD according to the methods of present invention in the same laboratory, or the cells may be delivered to a second location for PGD. If PGD is performed at a different location than where the embryo is maintained, PGD results may be transmitted back to the laboratory maintaining the embryo where transfer of suitable embryos into the host may then be performed. As would be apparent to one of skill in the art, the steps of the method are ideally performed in locations such that the entire process takes place in the most efficient manner possible; for example, in one embodiment, the IVF embryo is maintained in the same facility in which the genetic screening is performed and in which embryo transfer takes place. In this way, loss of time associated with shipping the cells to a second laboratory for genetic analysis is avoided. This would be especially advantageous where the time available for performing PGD and embryo transfer is extremely limited, for example, with regard to the same day biopsy, PGD and fresh transfer of a day 5 or day 6 blastocyst.

As contemplated herein, genotypic analysis of an “embryo” includes assay of nucleic acid from cells from an IVF embryo (an embryo fertilized not less than about 40 hours before genotyping), cells from a blastocyst (typically an embryo at day 4, day 5 or day 6 after fertilization) as well as cells biopsied from an embryo but of extraembryonic origin, e.g., trophectoderm, or polar bodies. The plural form of this term is included, such that, the term “an embryo” as used herein contemplates that more than one embryo or blastocyst may be concurrently assayed or transferred according to the methods of the present invention.

It is further contemplated herein that more than one cell of an embryo may be biopsied as conditions permit, for example, one or more cells of trophectoderm may be biopsied and assayed according to the methods of the present invention. Assaying more than one cell in this way can be used to detect mosaicism in an embryo (a condition in which cells in an embryo may differ genetically from other cells in the embryo) which cannot be detected if only a single cell is assayed. Thus, as contemplated herein, the methods of the present invention can be used to biopsy a day 5 or day 6 embryo, screen the embryo for mosaicism, and still permit fresh transfer of the embryo on the same day.

As used herein, “genotyping” an informative SNP or a potentially informative SNP refers to analyzing the nucleic acid of a SNP to determine the DNA sequence information for that SNP.

“Transferring” an IVF embryo refers to the process of placing an IVF into a female patient with the objective that the embryo will implant and result in a viable pregnancy.

The term “fresh transfer” refers to the transfer of an embryo which has not been subjected to cryogenic preservation.

As used herein, a “plurality of chromosomes” refers to more than one chromosome.

“Candidate IVF embryos” are those embryos determined to be without genetic defect according to the methods of the present invention. These embryos may be deemed suitable for transfer, however, it is understood that other criteria familiar to one of skill in the art may also be taken into consideration in the selection of particular embryos for transfer.

The terms “chromosomal abnormality” and “genetic defect” are used interchangeably herein and refer to a deviation between the structure of the subject chromosome and a normal chromosome. The term “normal” refers to the predominate karyotype or banding pattern found in healthy individuals of a particular species. A chromosomal abnormality or genetic defect can be numerical or structural, and includes but is not limited to, single gene defects, sex-linked disorders, or chromosomal disorders, e.g., aneuploidy, polyploidy, inversion, a trisomy, a monosomy, duplication, deletion or additions of entire chromosomes or parts thereof, insertions, rearrangements, and translocations.

A chromosomal abnormality or genetic defect can be correlated with presence of a pathological condition or with a predisposition to develop a pathological condition. Numerous examples of pathological conditions associated with genetic defects on particular chromosomes and/or linked to a particular gene are known to those of skill in the art and literature references and electronic databases containing extensive and detailed information describing genetic defects are widely available. Such conditions, include, but are not limited to, the genetic diseases listed herein in Table 1 and as described in U.S. Pat. No. 7,439,346.

TABLE 1
Examples of Genetic Diseases

| Chromosome Number | Genetic Disease |
|---|---|
| 13 | Breast and ovarian cancers, |
| | deafness, Wilson's Disease |
| 15 | Marfan Syndrome, Tay-Sach's Disease |
| 16 | Polycystic kidney disease, Alpha |
| | thalassemia |
| 17 | Charcot-Marie-Tooth Disease |
| 18 | Niemann-Pick Disease, pancreatic |
| | cancer |
| 21 | Down's Syndrome |
| X | Duchenne muscular dystrophy (DMD), |
| | Turner's Syndrome, Fragile X |
| | Syndrome |
| | X-linked diseases: hemophilia, |
| | adrenoleukodystrophy, and Hunter's |
| | disease |
| Y | Acute myeloid leukemia |

As contemplated herein, disorders with genetic origins other than a SNP (i.e., microdeletions, translocations, or rearrangements) can also be screened using targeted preamplification strategies and in parallel with SNP based aneuploidy screening. In addition, one can employ the methods of the present invention to concurrently screen an IVF embryo for aneuploidy as well as for one or more single gene genetic disorders. For example, if a single gene disorder were identified in the prospective parents, that disorder could be evaluated in parallel with aneuploidy screening by co-amplifying the disease causing target sequence or linked DNA sequences with the aneuploidy informative SNPs. Analysis of the aneuploidy state of each chromosome and the presence or absence of the single gene disorder could then be evaluated in each resulting embryo. In this way, simultaneous analysis of any single gene disorder or other genetic disorder (microdeletions, translocations, rearrangements, etc.) could be evaluated in parallel with 24 chromosome aneuploidy screening.

As used herein, an IVF embryo “determined to be without genetic defect” refers to an embryo that is determined to be free of a particular genetic defect for which it was screened. It is understood that while the methods of the present invention are accurate, they may not be able to detect 100% of the genetic abnormalities in an IVF embryo. In some cases, data may be interpreted as meaning that there is a greatly reduced chance of the IVF embryo having a particular genetic defect, e.g., as would be the case for diagnosing mosaicism in an embryo, given that only a few cells, at best, may be assayed.

As contemplated herein, to improve the accuracy of the data obtained, the methods of the present invention may involve determining the sequence of multiple alleles of interest on a single chromosome. For example, the sequence of one to tens to hundreds to thousands of potentially informative SNPs on a single chromosome may be determined. In addition, the sequence of multiple alleles of interest on multiple chromosomes may be determined.

Standard techniques for nucleic acid isolation and purification are known and are described in, for example, in Miller (ed.) 1972 Experiments in Molecular Genetics, Cold Spring Harbor Laboratory, Cold Spring Harbor, N.Y.; Old and Primrose, 1994 Principles of Gene Manipulation, 5th ed., University of California Press, Berkeley; Schleif and Wensink, 1982 Practical Methods in Molecular Biology; Glover (Ed.) 1985 DNA Cloning: Vols. I AND II, IRL Press, Oxford, UK; Harnes and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York City.

The sequence of a nucleic acid may be determined using conventional methods. These methods include, for example, PCR, gel electrophoresis, ELISA, mass spectrometry, MALDI-TOF mass spectrometry hybridization, primer extension, fluorescence detection, fluorescence resonance energy transfer (FRET), fluorescence polarization, DNA sequencing, Sanger dideoxy sequencing, DNA sequencing gels, capillary electrophoresis on an automated DNA sequencing machine, microchannel electrophoresis, microarray, southern blot, slot blot, dot blot, single primer linear nucleic acid amplification, as described in U.S. Pat. No. 6,251,639, SNP-IT, GeneChips®, HuSNP®, BeadArray, TaqMan° assay, Invader® assay, MassEXTEND®, or MassCLEAVE™ (hMC) method.

Nucleic acid amplification methods are also well known, including polymerase chain reaction (PCR) (PCR Protocols, A Guide to Methods and Applications, ed. Innis, Academic Press, N.Y. 1990; PCR: A Practical Approach, M. J. McPherson, et al., IRL Press (1991)); ligase chain reaction (LCR) (Landegren, Science 1988; 241:1077;); transcription amplification (Kwoh, Proc. Natl. Acad. Sci. USA 1989; 86:1173); self-sustained sequence replication (Guatelli, Proc. Natl. Acad. Sci. USA 1990; 87: 1874); Q Beta replicase amplification (Smith, J., Clin. Microbial. 1997; 35:1477-1491), and other RNA polymerase mediated techniques such as nucleic acid sequence based amplification, NASBA (U.S. Pat. Nos. 4,683,195 and 4,683,202); 3SR (self-sustained sequence reaction); RACE-PCR (rapid amplification of cDNA ends); PLCR (a combination of polymerase chain reaction and ligase chain reaction); SDA (strand displacement amplification); and SOE-PCR (splice overlap extension PCR). In a particular embodiment, the sample nucleic acid is amplified using PCR.

Errors associated with amplification include allelic, or locus dropout (LDO) and such errors are familiar to one of skill in the art. LDO rate estimates can differ based on the method by which they are measured. For example, microarrays often underestimate LDO rates. There are analysis settings that can be adjusted that will lead to different estimates, e.g., stringent genotype calls are associated with higher LDO rate estimates. Real time PCR is the most stringent method for evaluating LDO so estimates based on the use of real time PCR will be higher than microarray measures.

In order to avoid misdiagnoses due to amplification errors such as allelic or locus drop out, it is understood herein that sequencing an allele of interest may include sequencing nucleic acid around the allele to ensure amplification accuracy. For example, the disease-causative allele may be physically linked (close together) with a non-causitive allele nearby in the DNA sequence. These two sites in the DNA are very likely to be inherited together, barring any meiotic recombination between the sites. Sites nearer each other are less likely to undergo recombination. As a result, the non-causative allele can be used as a confirmatory marker of the disease causing allele in order to avoid misdiagnosis from disease allele PCR dropout. Such techniques are familiar to one of skill in the art.

It is contemplated herein that conventional methods to analyze embryonic and parental nucleic acid include methods that permit the-analysis of nucleic acid from a small number of cells. Such methods may include performing a “preamplification” of DNA prior to real-time PCR using SNP locus specific primers. Such methods are a modification of methods familiar to one of skill in the art, and kits to perform such preamplification are commercially available, for example, TaqMan® PreAmp Cells-to-Ct™ Kit from Applied Biosystems. While these kits are designed to preamplify cDNA derived from RNA, they can also be used successfully on genomic DNA.

As contemplated herein, in a particular embodiment the methods of the present invention are performed using means which permit multiple, parallel real-time PCR reactions, including, but not limited to, high throughput genotyping using one or more assay platforms. By evaluating multiple alleles of interest in this way, preimplantation genetic diagnosis may be performed quickly and efficiently such that embryo genotyping and transfer may occur without the need for cryopreservation of the embryo, ideally, such steps occur in the same day. For example, it is contemplated herein that candidate embryo selection and embryo transfer may be performed within about 8 hours after biopsy of the embryo for genotyping. It is further contemplated herein that the robust methods of the present invention may permit the genetic screening of all 24 chromosomes of an IVF embryo followed by fresh transfer of the embryo on the same day.

Materials and methods for high throughput real-time PCR are familiar to those of skill in the art. These include, but are not limited to, commercial genotyping plates designed for such purposes. For example, high throughput methodologies which may be employed to practice the methods of the present invention include commercially available microarray plates that use nanoliter fluidics, for example, Applied Biosystems' TaqMan® OpenArray™ Genotyping Plates; which may be customized to contain nucleic acid for alleles of interest. Additional systems include the Fluidigm Inc. EP1 System for genetic analysis.

Primers for use in the methods of the present invention may be obtained from commercial vendors. They may also be designed by one of skill in the art according to conventional methods and published sequence information for any SNP of interest.

Each SNP specific PCR reaction may include allele specific probes with unique fluorescent properties. Reaction products will result in a different wavelength of quantifiable fluorescence. The alleles of interest can be analyzed using conventional gel electrophoresis followed by fluorescence detection or read using a commercially available fluorescent plate reader or scanner. Commercially available computer imaging systems designed for high throughput include, e.g., OpenArray™ NT Imager (Applied Biosystems Inc.), EP1 reader (Fluidigm, Inc.), or the LightCycler® (Roche). The resulting fluorescence data can be evaluated with statistical protocols and computer programs such as GENESCAN (Applied Biosystems), IMAGEQUANT (GE Healthcare), and OpenArray™ (Applied Biosystems) software to determine allele ratios for PGD. (See, e.g., Livak, K. L. and Todd, J. A., Nature Genetics Vol. 9, April 1995, 341-342).

As contemplated herein, the methods of the present invention may be used to screen one or more candidate IVF embryos concurrently such that more than one IVF embryo without genetic defect may be identified and transferred. The number of such embryos that may be appropriate to transfer may be determined by one of skill in the art according to conventional methods.

All publications cited in the specification are indicative of the level of skill of those skilled in the art to which this invention pertains. All these publications are herein incorporated by reference in their entirety to the same extent as if each individual publication were specifically and individually indicated to be incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### EXAMPLES

### Example 1

### Aneuploidy Detection Based on Genotyping Informative SNPs in Single Cells From a Trisomy 21 and a Monosomy 21 Cell Line

We established the feasibility of the techniques described in this application in a series of experiments. Experiment 1 can be divided into three steps:

Step 1. Identification of Informative SNPs.

In this example, we identified informative SNPs in cell lines with aneuploidy or euploidy of chromosome 21. To do this, we isolated total DNA from 4 cell lines (Coriell Cell Repository) known to have either a trisomy 21 (48, XY, +16, +21; ID# GM04435), a monosomy 21 (45, XX, −21; ID#GM01201), or a normal number of chromosome 21 (47, XY, +13; ID#GM02948, and 47, XY, +18; ID#GM01359). Microarray data was generated to identify SNPs with genotypes for the 4 samples which met the following criteria. We looked for SNPs that were heterozygous in the trisomy 21 cell line, homozygous in the monosomy 21 cell line, heterozygous in one of the two normal chromosome 21 cell lines, and homozygous for the opposite allele as the monosomy 21 cell line in the other of the two normal chromosome 21 cell lines. We selected 10 SNPs which met these criteria (Table 2). Selection of informative SNPs would be performed in a different manner in the clinical PGD situation where parental DNA is evaluated as described above.

Step 2. Evaluate Informative SNPs in Single Cells.

Using conventional methods, we obtained single cells from each of the 4 cell lines by removal from culture media using a dissecting microscope and stripper tip pipet. Single cells were placed into a nuclease- and nucleic acid-free 0.2 ml PCR tube in a 2 microliter (μl ) volume. Alkaline lysis was performed and the sample lysate was stored at −20° C. The lysate was then used to preamplify the 10 target regions of interest in the genome where the selected informative SNPs are found. This is done by adding a solution that contains a pool of primer sets (TaqMan SNP Genotyping Assay IDs in Table 2) for each region of interest to the lysate along with a PCR buffer and DNA polymerase such as the PreAmp Master Mix (Applied Biosystems, Inc.). Once the mixture is set up, the reaction is then run on a thermalcycler for 14 cycles (95° C.-10 min, then 14 times of 95° C.-15 sec, 60° C.-4 min, then hold at 4° C.). The resulting preamplified DNA is then aliquoted and mixed with reagents for multiple, separate, and parallel, 5 μl PCR reactions for each specific SNP on a 384 well plate. Each SNP PCR reaction is a duplex 5′ nuclease assay that includes allele specific probes with unique fluorescent properties. PCR products from each allele result in a different wavelength of quantifiable fluorescence. These conditions would likely need to be optimized for analysis of embryonic biopsy tissue but the overall concept of preamplification followed by PCR and end point quantification of allele specific fluorescence intensity is applicable.

Step 3. Analysis of Data.

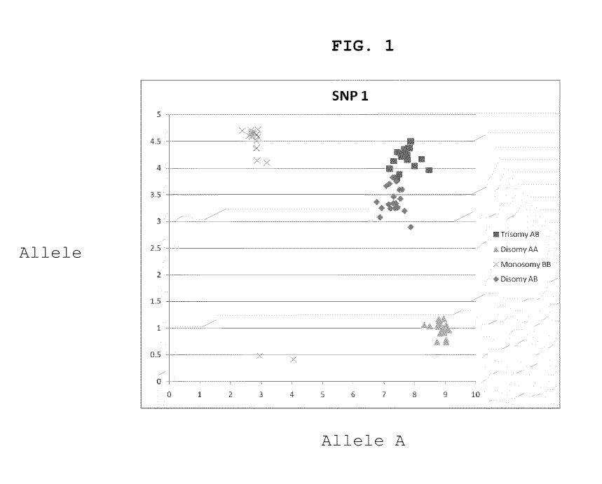

In this example, the resulting fluorescence data was exported from the data acquisition software (SDS 2.3, Applied Biosystems Inc.) as a text file and opened with Microsoft Excel to generate graphical representations (see, e.g., Livak, K. L. and Todd, J. A., Nature Genetics Vol. 9, April 1995, 341-342, the entire contents of which are incorporated by reference and is attached herein as part of this application). Plots of each SNP were generated in particular to test whether the trisomy 21 cells' heterozygous data points were distinguishable from the normal 21 cells' heterozygous data points (FIG. 1). By definition, informative SNPs for monosomy are restricted to SNPs for which the parents are homozygous for the opposite allele. In the situation of monosomy, the genotype call is homozygous and is therefore much more distinguishable from the expected heterozygous genotype then would be a trisomy derived heterozygous genotype. Nonetheless, the example shown in FIG. 1 illustrates the capability of distinguishing trisomy heterozygous from disomy heterozygous. This is a direct result of the presence of a 2:1 allele ratio in the trisomy 21 cells and a 1:1 ratio in the normal 21 cells.

TABLE 2
Genotypes of 10 SNPs in 4 cell lines used to establish feasibility of this invention

| TaqMan SNP | Affymetrix | | Normal | Normal | Monosomy | Trisomy |
|---|---|---|---|---|---|---|
| Genotyping | Nspl Array | dbSNP | 21 | 21 | 21 | 21 |
| Assay ID | SNP ID | RS ID | (GM01359) | (GM02948) | (GMA1201) | (GM04435) |
| C_3270895_1_{—} | SNP_A- | rs2280956 | BB | AB | AA | AB |
| | 2020731 | | | | | |
| C_2817907_1_{—} | SNP_A- | rs2823976 | BB | AB | AA | AB |
| | 4208962 | | | | | |
| C_2871960_1_{—} | SNP_A- | rs2830169 | BB | AB | AA | AB |
| | 2017082 | | | | | |
| C_3270619_10 | SNP_A- | rs2830705 | BB | AB | AA | AB |
| | 1969727 | | | | | |
| C_3270620_10 | SNP_A- | rs2830706 | BB | AB | AA | AB |
| | 4209062 | | | | | |
| C_2439141_1_{—} | SNP_A- | rs2835795 | BB | AB | AA | AB |
| | 2019020 | | | | | |
| C_2602571_1_{—} | SNP_A- | rs2837774 | BB | AB | AA | AB |
| | 1970468 | | | | | |
| C_3270928_1_{—} | SNP_A- | rs2839171 | BB | AB | AA | AB |
| | 2020733 | | | | | |
| C_3270837_1_{—} | SNP_A- | rs2968 | BB | AB | AA | AB |
| | 2020720 | | | | | |
| C_1211578_1_{—} | SNP_A- | rs914210 | BB | AB | AA | AB |
| | 2020579 | | | | | |

### Example 2

### Single Gene Genetic Disorders Detected Based on Presence or Absence of SNP Allele

There are many types of genetic mutations that cause disorders in humans. Many single gene disorders are caused by inheritance of a causative SNP allele. For example, a transition of nucleotide G to A at position 1138 of the fibroblast growth factor receptor 3 gene (FGFR3) results in an amino acid change of Glycine to Arginine at position 380 in the FGFR3 protein, and is the major cause of dwarfism. Therefore, in the case where patients carrying the A SNP allele would prefer to prevent transfer of embryos with this mutation, the presence of A would be evaluated from embryo biopsy DNA. A SNP genotyping assay for position 1138 of FGFR3, would be performed with the outcome being presence or absence of the A allele. We tested this concept by obtaining cell lines from a family that carries this particular FGFR3 polymorphism. The parents of the affected child were both heterozygous for the dwarfism allele (genotype GA) and had cell lines available for analysis through the Coriell Cell Repository (IDs GM08858 and GM08857). The affected child was homozygous for the dwarfism allele (genotype AA) and had a cell line available for analysis (GM08859). As a control, we also obtained a cell line (GM18666) which was homozygous for the normal allele (genotype GG). Two sets (biological replicates) of five cells (representing a quantity of cells typically obtained from a trophectoderm biopsy) from each cell line were processed as described in Example 1. The preamplification and PCR steps were performed using a custom designed TaqMan SNP Genotyping Assay using the sequences shown below as SEQ ID NO:1 and SEQ ID NO:2. Probes were made complimentary to one or the other sequences containing either allele G or A (noted in bold font). Each probe is labeled with a unique fluorophore (e.g., VIC or FAM) so that if one or both alleles are present in the sample, one will observe fluorescence from one or both dyes.

(SEQ ID NO: 1)

CTAGACTCACTGGCGTTACTGACTGCGAGACCCTCCAGACAAGGCGCGTG
CTGAGGTTCTGAGCCCCCTTCCGCTCCCAGTGGTGCCTGCGGCTCTGGGC
CAGGGGCATCCATGGGAGCCCCGTGGNGGGGGGGNCCAGGCCAGGCCTCA
ANNCCCATGTCTTTGCAGCCGAGGAGGAGCTGGTGGAGGCTGACGAGGCG
GGCAGTGTGTATGCAGGCATCCTCAGCTACGGGGTGGGCNTCNTCCTGTT
CATCCTGGTGGTGGNGGCTGTGACGCTCTGCCGCCTGCGCAGCCCCCCCA
AGAAAGGCCTGGGCTCCCCCACCGTGCACAAGATCTCCCGCTTCCCGCTC
AAGCGACAGGTAACAGAAAGTAGATACCAGGTTCTGAGCTGCCTGCCCGC
CAGGCCTCCTGGAGCCCCACCTCGGCCCACGCTGGTCCTGGGCTGTGTGA
GCCCTCTCTGCAGCCAGGCGGGCTCCCCTCTCCTCGTCTCTGNTCACCAT
GTAGAGCCTAGGGTAC

(SEQ ID NO: 2)

CTAGACTCACTGGCGTTACTGACTGCGAGACCCTCCAGACAAGGCGCGTG
CTGAGGTTCTGAGCCCCCTTCCGCTCCCAGTGGTGCCTGCGGCTCTGGGC
CAGGGGCATCCATGGGAGCCCCGTGGNGGGGGGGNCCAGGCCAGGCCTCA
ANNCCCATGTCTTTGCAGCCGAGGAGGAGCTGGTGGAGGCTGACGAGGCG
GGCAGTGTGTATGCAGGCATCCTCAGCTACAGGGTGGGCNTCNTCCTGTT
CATCCTGGTGGTGGNGGCTGTGACGCTCTGCCGCCTGCGCAGCCCCCCCA
AGAAAGGCCTGGGCTCCCCCACCGTGCACAAGATCTCCCGCTTCCCGCTC
AAGCGACAGGTAACAGAAAGTAGATACCAGGTTCTGAGCTGCCTGCCCGC
CAGGCCTCCTGGAGCCCCACCTCGGCCCACGCTGGTCCTGGGCTGTGTGA
GCCCTCTCTGCAGCCAGGCGGGCTCCCCTCTCCTCGTCTCTGNTCACCAT
GTAGAGCCTAGGGTAC

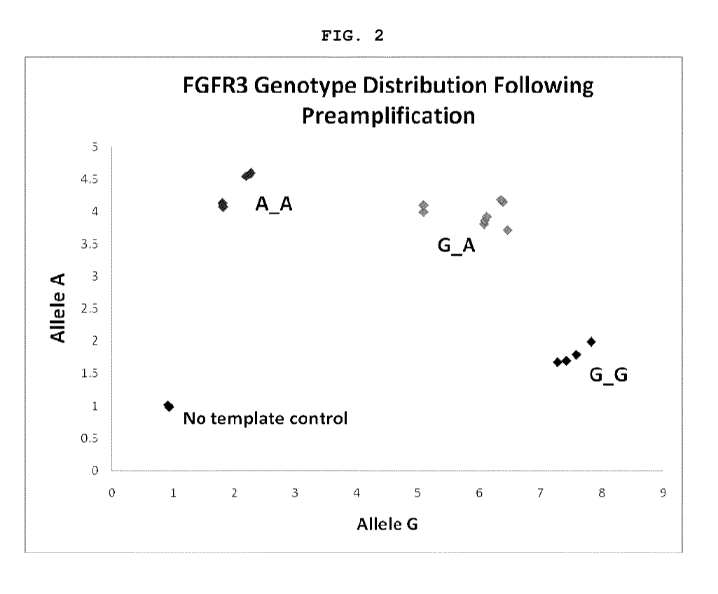

Results of running the assay were as expected (FIG. 2), demonstrating the detection of the presence of a homozygous dwarfism allele genotype in the cells from the affected child's cell line, heterozygous in the cells from both parental cell lines, and homozygous for the normal allele in the cells from the control cell line. These data in combination with Example 1 experiments, demonstrate the feasibility of evaluating single gene disorders in parallel with 24 chromosome aneuploidy on human embryo biopsy tissue.

In addition to evaluating the disease causing allele directly, a nearby informative SNP allele may be used to track the inheritance of the disorder-causing SNP allele. This procedure of evaluating multiple disease markers prevents misdiagnosis as a result of allelic dropout.

### Example 3

### Microdeletion Detected Based on Evaluation of Informative SNPs

Disorders with genetic origins other than a SNP (i.e., microdeletions, translocations, or rearrangements) can also be screened using similar preamplification strategies and in parallel with SNP based aneuploidy screening. This is simply done by coamplification of the informative aneuploidy screening SNPs with the disease DNA locus of interest.

For example, we identified a 2.36 MB microdeletion in a patient with Alagille syndrome by using SNP microarray analysis of genomic DNA isolated from the patient's blood. The microdeletion occurred within a region containing the Jagged 1 gene. Mutations and microdeletions involving the Jagged 1 gene are known to cause Alagille syndrome (Li et al., Nature Genetics 16:243-251 (1997); Oda et al., Nature Genetics 16: 235-242 (1997)). There were 309 SNPs within the patient's microdeletion. Forty four of these SNPs were homozygous opposite in the patient and her partner (for which whole blood isolated genomic DNA was also evaluated). We selected 8 of these informative SNPs to evaluate performance on 5-cell samples from the patient and her partner, either separate or mixed. Performance was as expected for 7 of the 8 SNPs, in that 5 cell samples from each parent were homozygous for the opposite allele, and mixed cells were heterozygous (Table 3). In embryos resulting from IVF treatment of this couple, one would expect normal embryos to possess a heterozygous genotype (one allele for each parent), and abnormal embryos to possess a homozygous genotype identical to the noncarrier partner.

By amplifying these 7 SNPs in parallel with aneuploidy informative SNPs, one could diagnose Alagille syndrome and 24 chromosome aneuploidy in parallel. The same concept applies to any possible inheritable genetic disorder.

TABLE 3
Genotyping results of candidate SNPs informative for inheritance of a microdeletion from an Alagille syndrome case

| Summary Sample | Type | SNP_1 | SNP_2 | SNP-3 | SNP_4 | SNP_5 | SNP_6 | SNP_7 | SNP_8 |
|---|---|---|---|---|---|---|---|---|---|
| Maternal Exp. | Microarray gDNA | A_A | A_A | C_C | T_T | G_G | T_T | A_A | A_A |
| Maternal Obs. | TaqMan gDNA | A_A | A_A | C_C | T_T | G_G | T_T | A_A | A_A |
| Maternal PreAmp 1 | 5-cell | A_A | A_A | C_C | T_T | G_G | T_T | A_A | A_A |
| Maternal PreAmp 2 | 5-cell | A_A | A_A | C_C | T_T | G_G | T_T | A_A | A_A |
| Maternal PreAmp 3 | 5-cell | A_A | A_A | C_C | T_T | G_G | T_T | A_A | A_A |
| Maternal PreAmp 4 | 5-cell | A_A | A_A | C_C | T_T | G_G | T_T | A_A | A_A |
| Paternal Exp. | Microarray gDNA | G_G | G_G | T_T | C_C | A_A | C_C | G_G | G_G |
| Paternal Obs. | TaqMan gDNA | A_G | G_G | T_T | C_C | A_A | C_C | G_G | G_G |
| Paternal PreAmp 1 | 5-cell | A_G | G_G | T_T | C_C | A_A | C_C | G_G | G_G |
| Paternal PreAmp 2 | 5-cell | A_G | G_G | T_T | C_C | A_A | C_C | G_G | G_G |
| Paternal PreAmp 3 | 5-cell | A_G | G_G | T_T | C_C | A_A | C_C | G_G | G_G |
| Paternal PreAmp 4 | 5-cell | A_G | G_G | T_T | C_C | A_A | C_C | G_G | G_G |
| 2:1 KS:MS | 5-cell mixture | NA | A_G | C_T | C_T | A_G | C_T | A_G | A_G |
| 2:1 gDNA KS:MS | 5-cell mixture | NA | A_G | C_T | C_T | A_G | C_T | A_G | A_G |
| Maternal Neg Control | blank | ND | ND | ND | ND | ND | ND | ND | ND |
| Paternal Neg Control | blank | ND | ND | ND | ND | ND | ND | ND | ND |
| NTC | blank | ND | ND | ND | ND | ND | ND | ND | ND |

### Example 4

### Amplification of Specific Sequences From Single Cells is More Successful Using a Targeted Approach Than Using Whole Genome Amplification

Single cells from a normal male cell line were subject to either whole genome amplification (WGA) or preamplification of the target sequences (Targeted Amp.) using conventional methods. Real-time PCR was then performed to evaluate the DNA for the presence or absence of Y chromosome target sequences. Drop out rate was calculated by dividing the number of failed amplifications (no fluorescence detected) by the total number of amplifications attempted. Data indicate the advantage of targeted amplification over whole genome amplification as the dropout rate associated with targeted amplification is significantly lower than that seen after whole genome amplification, thus allowing for more comprehensive analysis of the target sequences (see Table 4). In order to produce similar accuracy in overall aneuploidy diagnosis, WGA methods require higher throughput analysis such as a genotyping microarray would provide. However, this approach takes considerably longer time to perform than would be necessary with a targeted amplification strategy using real-time PCR. Thus, there is a considerable time advantage associated with amplification of only the informative SNP target sequences for PGD according to the methods of the present invention.

TABLE 4
Amplification of specific sequences from a single
cell using targeted approach (“Targeted Amp.”)
and whole genome amplification (“WGA”)

Method

| | Targeted Amp. | WGA |
|---|---|---|
| No. failed | 25 | 540 |
| amplifications | | |
| No. attempted | 351 | 1020 |
| amplifications | | |
| Dropout rate | 7% | 53% |

1. A method for determining the presence or absence of a genetic defect in an IVF embryo prior to transfer comprising:
(a) identifying a set of informative SNPs in the genotype of the embryo's parents;
(b) assaying the genotype of two or more informative SNPs from the set of informative SNPs on one or more chromosomes collected from a cell of the embryo;
(c) determining the presence or absence of a genetic defect in the embryo based on the genotype of the two or more informative SNPs on one or more chromosomes of the embryo; and
(d) selecting a candidate IVF embryo determined to be without genetic defect for transfer.

2. The method of claim 1 wherein the genetic defect is a mutation in the DNA of the embryo, a chromosomal abnormality and/or aneuploidy.

3. The method of claim 2 wherein the genetic defect is selected from the group consisting of those provided in Table 1.

4. The method of claim 2 wherein the aneuploidy is selected from the group consisting of nullisomy, monosomy, disomy, trisomy, and tetrasomy.

5. The method of claim 1 wherein the embryo is genotyped comprising using a subset of the informative SNPs identified in the genotype of the embryo's parents.

6. The method of claim 1 wherein the step of identifying the set of informative SNPs comprises determining one or more heterozygous SNPs on one or more chromosomes of the embryo's parents.

7. The method of claim 1, wherein the step of identifying the set of informative SNPs comprises determining one or more homozygous opposite SNPs on one or more chromosomes of the embryo's parents.

8. The method of claim 1 wherein determining the presence or absence of a genetic defect in the embryo comprises genotyping two or more informative SNPs on a plurality of chromosomes of the embryo.

9. (canceled)

10. The method of claim 1 wherein the IVF embryo is a human embryo.

11. The method of claim 1 wherein the IVF embryo is a day 3, day 4, day 5 or day 6 embryo.

12. (canceled)

13. (canceled)

14. The method of claim 1 further comprising transferring the selected candidate IVF embryo on the same day as the steps of assaying, determining and selecting.

15. The method of claim 14 wherein assaying, determining, selecting and transferring of the IVF embryo are accomplished within about 24 hours.

16. The method of claim 14 wherein assaying, determining, selecting and transferring of the IVF embryo are accomplished within about 12 hours or less.

17. The method of claim 14 wherein assaying, determining, selecting and transferring of the IVF embryo are accomplished within about 8 hours or less.

18. The method of claim 1 further comprising assaying more than one cell from the IVF embryo.

19. The method of claim 18 wherein the IVF embryo is a blastocyst.

20. The method of claim 19 wherein the cells are biopsied from trophoectoderm.

21. The method of claim 20 wherein assaying, determining, selecting and transferring of the blastocyst are accomplished within about 24 hours.

22. The method of claim 20 wherein assaying, determining, selecting and transferring of the blastocyst are accomplished within about 12 hours or less.

23. (canceled)

24. The method of claim 14 wherein three or less IVF embryos are transferred.

25. (canceled)

26. (canceled)

27. The method of claim 1 wherein the informative SNPs are identified comprising the use of a database of genetic information.

28. (canceled)

29. (canceled)

30. The method of claim 27, wherein the database contains information regarding diallelic SNPs with a high minor allele frequency.

31. The method of claim 30 wherein the high minor allele frequency is from about 40% to less than about 50%.

32. The method of claim 7, further comprising selecting about 1000 or less homozygous opposite SNPs from the set of informative SNPs to generate a subset of informative SNPs for genotyping the one or more chromosomes of the embryo.

33. (canceled)

34. (canceled)

35. (canceled)

36. (canceled)

37. (canceled)

38. The method of claim 1, wherein the subset of informative SNPs comprises about 100 or less SNPs per chromosome.

39. (canceled)

40. (canceled)

41. (canceled)

42. (canceled)

43. (canceled)

44. (canceled)

45. A method for transferring an IVF embryo comprising
(a) identifying informative SNPs by genotyping SNPs on a plurality of chromosomes of the parents to identify homozygous opposite SNPs;
(b) assaying the genotype one or more of the informative SNPs on a plurality of chromosomes collected from a cell of the embryo;
(c) determining the presence or absence of a genetic defect in the embryo based on the genotype of the informative SNPs in the embryo; and
(d) transferring the embryo if determined to be without genetic defect within about 154 hours of fertilization.

46. The method of claim 45 wherein the embryo is transferred between about 48 and about 144 hours of fertilization.

47. The method of claim 45 wherein the assaying, determining and transferring steps are accomplished within a period of about 48 hours.

48. The method of claim 45 wherein the assaying, determining and transferring steps are accomplished within a period of about 24 hours.

49. (canceled)

50. (canceled)

51. (canceled)

52. The method of claim 46 wherein the assaying, determining and transferring steps are accomplished within a period of about 48 hours.

53. The method of claim 46 wherein the assaying, determining and transferring steps are accomplished within a period of about 24 hours.

54. (canceled)

55. (canceled)

56. (canceled)

57. The method of claim 46, wherein only a subset of informative SNPs genotyped in the parents is genotyped in the embryo.

58. (canceled)

59. A method for preimplantation genetic diagnosis and fresh transfer of a day 3, day 4, day 5 or day 6 IVF embryo comprising
(a) identifying informative SNPs by genotyping SNPs on a plurality of chromosomes of the parents to identify homozygous opposite SNPs;
(b) assaying the genotype of one or more of the informative SNPs in the embryo on a plurality of chromosomes of the IVF embryo;
(c) determining the presence or absence of a genetic defect in the embryo based on the genotype of the informative SNPs in the embryo; and
(d) transferring the embryo if determined to be without genetic defect within about 24 hours of the assaying step.

60. An array of informative SNPs or potentially informative SNPs comprising a plurality of nucleic acid probes comprising nucleic acid for one or more SNPs from one or more human chromosomes wherein the SNPs are homozygous opposites of high minor allele frequency.

61. (canceled)

62. (canceled)

63. A method for making an array for preimplantation genetic diagnosis of an IVF embryo comprising (a) identifying an informative SNP for preimplantation genetic diagnosis, (b) selecting an informative SNP for at least one chromosomes, and (c) affixing nucleic acid probes for the informative SNP on a solid support.

64. A kit comprising an array of nucleic acid probes immobilized on a solid support, the array comprising nucleic acid probes for one or more SNPs from one or more human chromosomes wherein the SNPs are informative SNPs for determining the presence or absence of a genetic defect in an IVF embryo prior to transfer according to the method of claim 1.


SEQUENCE LISTING (2 sequences)

SEQ ID NO: 1 - DNA, 516 bp, Homo sapiens
  modified_base (127)..(127): a, c, t, g, unknown or other
ctagactcactggcgttactgactgcgagaccctccagacaaggcgcgtgctgaggttctgagcccccttccgctcccagtggtgcctgcggctctgggccaggggcatccatgggagccccgtggngggggggnccaggccaggcctcaanncccatgtctttgcagccgaggaggagctggtggaggctgacgaggcgggcagtgtgtatgcaggcatcctcagctacggggtgggcntcntcctgttcatcctggtggtggnggctgtgacgctctgccgcctgcgcagcccccccaagaaaggcctgggctcccccaccgtgcacaagatctcccgcttcccgctcaagcgacaggtaacagaaagtagataccaggttctgagctgcctgcccgccaggcctcctggagccccacctcggcccacgctggtcctgggctgtgtgagccctctctgcagccaggcgggctcccctctcctcgtctctgntcaccatgtagagcctagggtac

SEQ ID NO: 2 - DNA, 516 bp, Homo sapiens
  modified_base (127)..(127): a, c, t, g, unknown or other
ctagactcactggcgttactgactgcgagaccctccagacaaggcgcgtgctgaggttctgagcccccttccgctcccagtggtgcctgcggctctgggccaggggcatccatgggagccccgtggngggggggnccaggccaggcctcaanncccatgtctttgcagccgaggaggagctggtggaggctgacgaggcgggcagtgtgtatgcaggcatcctcagctacagggtgggcntcntcctgttcatcctggtggtggnggctgtgacgctctgccgcctgcgcagcccccccaagaaaggcctgggctcccccaccgtgcacaagatctcccgcttcccgctcaagcgacaggtaacagaaagtagataccaggttctgagctgcctgcccgccaggcctcctggagccccacctcggcccacgctggtcctgggctgtgtgagccctctctgcagccaggcgggctcccctctcctcgtctctgntcaccatgtagagcctagggtac